(19) United States
(12) Patent Application Publication
(10) Pub. No.: US 2004/0047900 A1
(43) Pub. Date: Mar. 11, 2004

(54) Treatment of posterior capsule opacification

(76) Inventor: Bruce Duncan Samuel Allan, London (GB)

Correspondence Address: Dickstein Shapiro Morin & Oshinsky, 41st Floor, 1177 Avenue of the Americas, New York, NY, 10036-2714, US

(21) Appl. No.: 10/450,166

(22) PCT Filed: Dec. 11, 2001
(86) PCT No.: PCT/GB01/05465

(30) Foreign Application Priority Data
Dec. 11, 2000 (EP) 00311037.6

(51) Int. Cl. A61K 39/395; A61L 15/00

(52) U.S. Cl. 424/445; 424/146.1

(57) ABSTRACT

Posterior capsular opacification is inhibited by administration of polymer, having immobilised on the surface, a ligand for a death receptor, preferably joined by a spacer into the lens capsule following cataract surgery. The ligand is preferably Fas ligand. A spacer is preferably polyethyiene glycol. The polymer preferably constitutes an intraocular lens.

The present invention relates to the treatment of posterior lens opacification, a complication arising after cataract surgery. In particular the invention relates to the prevention of the proliferation and migration of lens epithelial cells after such surgery by the attachment to surface of the intraocular lens (IOL) implanted during cataract surgery of a death receptor ligand. Preferably the ligand binds to Fas receptor.

Opacification of the natural lens of the eye—cataract—is the leading cause of visual impairment. Globally, 20 million people are bilaterally blind from cataract. With population aging, this is set to double by 2020¹. Cataract surgery is the most common elective operation. Over 175,000 procedures are performed each year in the UK alone.

A 4-5 mm disc shaped piece of the anterior lens capsule is removed routinely during cataract surgery (anterior capsulotomy). Ultrasonic liquifaction and aspiration of the opacified natural lens is performed through this circular opening, leaving the remainder of the capsule (the capsular bag) intact. A synthetic intraocular lens (IOL) is implanted within the capsular bag. A scarring reaction to the procedure, posterior capsule opacification (PCO), is the commonest complication of contemporary cataract surgery.

In the natural lens, the interior aspect of the anterior capsule is lined by a simple cuboidal epithelium, whereas the posterior capsule is acellular. PCO is mediated by proliferation, posterior migration, and fibroblastic transformation of lens epithelial cells (LECs) after surgery (Apple DJ, Solomon K D, Tetz M R. Posterior capsule opacification. Surv Ophthalmol 1992; 37: 73-116). A reliable method of LEC knock-out would prevent PCO. LEC knock-out would also help to preserve normal capsular tissue compliance, promoting the possibility of using flexible, “shape memory” IOL materials to restore natural accommodation—the ability to focus oh near objects without reading glasses (Allan B D S. Intraocular lens implants. BMJ 2000; 320: 73-74).

Overall, laser capsulotomy for PCO is required in 25% of patients within 2 years (Schaumberg D A, Dana M R, Christen W G, Glynn R J. A systematic overview of the incidence of posterior capsular opacification. Ophthlalmology 1998; 105:1213-21). New acrylic lens materials have reduced but not eliminated PCO. Laser capsulotomy rates associated with some contemporary acrylic IOLs may be as low as 5% (Ursell P G, Spalton D J, Pande M V, Hollick E J, Barman S, Boyce J, Tilling K. Relationship between intraocular lens biomaterials and posterior capsular opacification. J Cataract Refract Surg 1998; 24: 35260); but this intervention rate probably only reflects the advanced end of a spectrum of post surgical visual degradation associated with LEC proliferation. Also, the mechanism by which contact with newer IOL materials appears to induce regression of infiltrating LECs has not been elucidated (Duncan G. Lens cell growth and posterior capsule opacification: in vivo and in vitro observations. Br J Ophthalmol 1998; 82:1102-1103).

A variety of pharmacological approaches to LEC knock-out based on cytotoxic drug delivery have been explored. These include intraoperative injection of ricin conjugated monoclonal antibodies directed against LEC epitopes (Tarsio J F, Kelleher P J, Tarsio M, Emery J M, Lam D M. Inhibition of cell proliferation on the lens capsule by 4197×-ricin A immunoconjungate. J Cat Refract Surg 1997; 23: 26066), and postoperative diffusion of thapsigargin from polymethylmethacrylate lens surfaces (Duncan G, Wormstone I M, Liu CSC. Thapsigargin coated intraocular lenses inhibit human lens epithelial cell growth. Nature Med 1997; 3: 1026-1028). An inescapable problem with any fluid phase approach is the potential for collateral damage caused by the drug fraction not delivered to the lens epithelium.

In cataract surgery and a number of other applications, including wound dressings and dressings to treat epithelial cancers, it may be desirable to modify a material surface so as to cause cell death in contacting tissues or body fluids.

It is known that some materials are resistant to adhesion and growth of cells. IOL surfaces have been treated to control cell adhesion and biocompatibility. Heparin has been attached to substrates including IOLs to increase resistance of the substrate to the attachment and activation of platelets. It is known that to attach antibodies and other specific binding proteins, including biotin and avidin, to surfaces or soluble polymer substrates, can confer desirable properties on the substrate. It is also known that it may sometimes be desirable when attaching peptide or protein ligands to space the ligand from the substrate to which it is attached, in order to leave the ligand active site free to interact with its receptor. Polyethylene glycol (PEG), other synthetic polymers (which includes oligomers), and oligopeptides have been used as spacers. Ligands have also been attached to synthetic polymers for use in affinity chromatography.

Apoptosis is a cell suicide mechanism by which cells undergo programmed death by a highly orchestrated system, in which the cell components are packaged for removal without causing inflammation. Many cells express, or can be induced to express, a group of transmembrane proteins known as death receptors (DRs). Binding of DRs with their specific extracellular ligands (DRligands), leads to transmembrane signalling to the intracellular apoptotic machinery, initiating apoptosis. Many tumour cells express DRs, but chemotherapy with DRligands has been too toxic to normal cells, many of which also express DRs constitutively, to be clinically useful.

One of the best characterised DRs is Fas, also known as CD95 or Apol. Antibodies to the extracellular domain of Fas (Fas Ab) and Fas ligand are commercially available from UpState Biotechnology Alexis Biochemicals and DNAX Research Institute of Molecular & Cell Biology. One FasAb is pentameric IgM and acts as a functional Fas ligand, activating apoptosis in cell lines known to express Fas. Fas is widely expressed in the anterior eye (Wilson S E, Li Q, Weng J, Barry-Lane P A, Jester J V, Liang Q, Wordinger R L. The Fas-Fas ligand system and other modulators of apoptosis in the cornea. Invest Ophth Vis Sci 1996; 37: 1582-1592), and TNF induced apoptosis has been identified in the developing chick lens (Wride M A, Sanders E J. Nuclear degeneration in the developing lens and its regulation by TNFalpha. Exp. Eye Res 1998; 66: 371-383). Deficient cellular protection from a variety of environmental insults may predispose to cataract development in association with LEC apoptosis (Li D W-C, Kuszak J R, Dunn K, Waan R-R, Ma W, Wang G-M et al. Lens epithelial apoptosis appears to be a common cellular basis for non-congenital cataract development in humans and animals. J Cell Biol 1995; 130: 169-181 and Harocopos G J, Alvares K M, Kolker A E, Beebe D C. Human age related cataract and lens epithelial cell death. Invest Ophth Vis Sci 1998; 39: 2696-2705).

Nishi, O. et al in Atavashii Ganka (1998) 15/10), 1445-1450 investigate transcription of apoptotic factors including Fas, Fas ligand and intracellular apoptosis modulators, in cataractons lens epithelial cells removed during cataract surgery. They also investigate the effect of Fas-stimulating Ab on LEC apotosis showing that it induces apoptosis and speculate that it has potential to prevent PCO.

However apoptotic signalling in LECs after cataract surgery remains unexplored.

In EP-A-0716095 antibodies are raised against the intracellular domain of Fas. The antibodies are used in a sandwich ELISa by using a commercially available monoclonal antibody against the extracellular domain of Fas (DX-3 from DNAX Research Institute) immobilised on the plate, a sample believed to contain Fas and the antibodies against the intracellular domain of Fas.

According to the invention there is provided a new use of a product comprising death receptor ligand covalently bound to a polymer in the manufacture of a composition for use in the treatment of a human or animal In the new use, the polymer is preferably at the surface of a solid substrate. For instance the polymer may form the bulk material of the substrate, or may be a coating, preferably a stable coating, on a body formed of another material. The invention may also have use where the polymer is in dissolved or suspended form in a fluid, for instance a liquid.

A surface to which the ligand is attached is generally, in the method of treatment, in contact with cells expressing the corresponding death receptor at their surface. The ligand has a specific recognition site for the extracellular domain of the death receptor. The ligand should be bound to the polymer in a manner allowing activation of the death receptor. The ligand may be an antibody, for instance an IgG or an IgM. Preferably it is a naturally occurring DR ligand, or DR activating fragment or derivative thereof. Suitably the cells are epithelial cells, for instance, lens epithelial cells, skin epithelial cells, especially cells of skin tumours.

According to a further aspect of the present invention there is provided a DRligand covalently bound to a polymer, preferably through a spacer.

A surface carrying the ligand is preferably one or both surfaces of an intraocular lens or another intraocular device. Where the device is in the path of light in the capsule, the substrate polymer should be sufficiently transparent to radiation, including visible light. Where the substrate is a lens it should have a suitable refractive index in situ. The polymer should preferably be in contact with the interior surface of the capsular bag to form a continuous ring of interfacial contact, preferably around the equatorial zone. The polymer may comprise the haptic of a lens forming a continuous ring for contact with the interior surface of the capsular bag, or it may be a capsular tension ring which is inserted into the capsular bag and provides anatomical support for weak or damaged lens capsules around the equatorial zone. Alternatively the polymer may form a lining for the lens capsule and be formed by introducing a curable liquid which cures in situ to form the polymer and provides an overall interface for the capsule inner surface and derivatised polymer. Alternatively, the polymer may be a gel or elastomeric material used to form a “phaco-ersatz lens” (Parel, J M et al., Graefe's Arch. Clin. Exp. Ophthalmol. (1986) 224, 165 et seq) which substantially fills the capsule. Suitable polymers are silicone oils and elastomers cross-linked hydrophilic polymers such as naturally occurring polymers, polyurethanes, hydrophilic and hydrophobic polyacrylic compounds. The DRligand should, in all these applications, be substantially permanently bound to (or immobilised on) the polymer surface, that is bound through biologically stable bonds, and prevented from cellular uptake.

The surface may be another implant or prosthesis or other medical device to be used in contact with biological fluid, or with an organ of a mammal, for instance a drug delivery device, a wound dressing, an anti-tumour implant, etc.

In the present invention the DRligand may be a ligand capable of binding to the extracellular binding domain of any known DR, or indeed a DR discovered subsequent to the filing date of the present application. In this invention a death receptor, DR, is a cell surface receptor which has direct access to the cell's apoptotic machinery. They activate death caspases upon appropriate binding at the cell surface by ligands. Preferably the ligand is suitable for binding to a DR selected from tumour necrosis factor receptor; avian CAR1 (Brojatsch et al. 1996); death receptor 3 (DR3 also known as Apo3, WSL-1, TRAMP, or LARD (Chinnaiyan et al. Science (1996) 274, 990 et seq.); death receptor 4, DR4 (Pan et al., Science, (1997) 276, 111 et seq.); death receptor 5 (DR5, also known as Apo2, TRAIL-R2, TRICK2 and KILLER) (Pan et al. Science (1997) 277, 815 et seq.), and most preferably Fas. The ligand is thus preferably a compound belonging to the TNF superfamily. Preferably the ligand is selected from FasL (also known as CD95L), Fas antibodies which activate Fas and active fragments thereof, TNF, lymphotoxin a, Apo3L (also known as TWEAK Marsters et al. Curr. Biol. (1998) 5, 525 et seq.) and Apo2L (also known as TRAIL, Wiley et al. Immunity, (1995) 3, 673 et seq.), as well as active fragments and/or derivatives of any of these ligands which are capable of binding to and activating the respective receptors.

Our work has shown that apoptosis of lens epithelial cells of one cell line is induced by Fas antibody. Previous work has shown TNF receptor expression in embryonic chick lenses (Wride et al., op. cit.). Where the device is an IOL, the ligand is preferably one known to bind to such DRs. Most preferably for such a device the ligand is FasAb or Fas ligand.

In order to optimise the activity of the bound ligand, it may be desirable for it to be joined through a spacer to any surface or other polymer substrate and for any linkage to be distant from the active binding site of the ligand. A spacer may be an oligo peptide linker, or preferably a synthetic polymer linker, especially a hydrophilic polymer, such as formed of polyethylene glycol, for instance having a molecular weight in the range 200 to 20,000 D, preferably in the range 500 to 5000 D.

Functional PEGs are commercially available. Functionalities are available for conjugation to various substrate groups. Heterobifunctional PEGs are available which may be used as starting materials. The two functionalities may be used for sequential reaction with the ligand and the surface. Where the functional PEG is first reacted with the ligand conjugates of biomolecules with PEG based spacers are formed which are intermediates useful for subsequent linkage to the polymer. The reactive conjugate is the reactive intermediate formed from such a heterobifunctional PEG and the ligand, and which has a further functionality for reaction with the substrate usually after deprotection and/or activation.

Preferably the polymer is reacted first with the bifunctional spacer precursor and the polymer-spacer conjugate then reacted with the ligand.

Functional groups which may be used to conjugate PEGs or other polymers to a DRligand are, for instance, hydroxyl, thiol, amino, activated amino, carboxyl, activated ester, imides of carboxylic groups, benztriazole carbonate, glycidyl ether, oxycarbonylimidazole, p-nitrophenylcarbonate, aldehyde, isocyanate, N-maleimido, vinylic, etc. Groups on the ligand with which these groups react are usually amino, carboxyl, hydroxyl or thiol groups. The reactions are carried out under suitable conditions such as are used in the conjugation of PEGs to biomolecules, with appropriate protection of other potentially reactive groups, to prevent by-product formation.

The functional group on the polymer surface which reacts with the DR ligand, the reactive group on an intermediate conjugate or a spacer precursor, as the case may be, may be naturally present on the polymer, or introduced by a preactivation step. Preactivation steps may involve gamma, u.v., radio frequency, plasma, glow discharge or electron beam irradiation, optionally followed by reaction with an activating agent, for instance a graftable compound, for instance using techniques based on U.S. Pat. No. 4,806,382, U.S. 5,326,584 or U.S. 5,376,400. Using the process of U.S. Pat. No. 5,376,400, for instance, a polymer surface formed of polymethylmethacrylate is treated by glow discharge plasma, to generate a site for initiation of radical polymerisation of ethylenically unsaturated compounds. A suitable ethylenically unsaturated compound for use in the present invention would be either a monoacrylated or monovinylic polyethyleneglycol, having at the end of the molecule distant from the unsaturated group a reactive group suitable for subsequent reaction with DR ligand, or a ligand preconjugated to mono-unsaturated PEG. Polyethylene oxide molecules have also been attached to polymer surfaces being IOL's by grafting ethylenically unsaturated primary amine group-containing monomers onto the surface of the IOL and reacting aldehyde-terminated PEG with the resultant surface. Other ways of activating a surface involve exposure to hydroxyl ions in water vapour plasma contact procedures to add hydroxyl groups, or treatment with cerium IV ions to form radicals.

FasAb has reactive pendant groups in the Fc portion which may be derivatised by reactive oligomers or polymers to form biostable peptide bonds, and to leave the active Fas binding sites free for interaction with Fas.

Naturally occurring Fas ligand, or a derivative thereof is preferably linked via the C-terminal residue, usually through the carboxyl group.

In the invention the DRligand when conjugated to the polymer will be capable of interacting with and activating the respective DR expressed on the surface of cells in vivo in the animal to which the conjugate is administered. Where, as in the preferred embodiment, the ligand is bound to the surface of an IOL, the binding of the ligand to DR expressed on the surface of lens epithelial cells will cause apoptosis, thereby preventing posterior capsular opacification. Further embodiments designed to control scarring reactions to cataract surgery may include binding of the DRligand to a prosthetic interior lining for the lens capsule an intracapsular ring, or a flexible IOL designed to preserve accommodation (Parel J-M, Gelender H, Trefers W F, Norton E W D. Phaco-Ersatz: cataract surgery designed to preserve accommodation Graefe's Arch Clin Exp Ophthalmol 1986; 224: 165 et seq). In another embodiment the substrate may be a wound dressing or a dressing for an epithelial cancer or a dressing for other hyperproliferative conditions such as excema or psoriasis, or a dressing for a viral lision especially a viral skin lesion. Provision of the ligand at the surface of the dressing should preferentially kill tumour cells or inflammatory cells which are known to express DRs. The advantage in immobilising the DR ligand to the surface is that this localises the ligand to the region of the body where cells which are required to apoptose are located. The immobilisation prevents delivery of the ligand to other parts of the body where undesirable toxic side effects might take place.

The polymer to which the ligand is attached is generally formed of synthetic polymer, although in some embodiments naturally-occurring polymers or their derivatives, may be used. Naturally-occurring polymers which may be used are for instance polysaccharides, proteins, nucleic acids, or combinations thereof. Synthetic polymers may be any of those used in medical devices, preferably biostable (as opposed to biodegradable or bioerodable) polymers. Examples are silicones, polyurethanes, polyolefins, polyesters, polyamides, polyethers, polyacrylic compounds or combinations of these. IOLs are usually made from silicones, or polyacrylate materials. The ligand may be incorporated into the polymer by using a polymerisable ligand as one of the monomers for forming the polymer, or, alternatively, by derivatising preformed polymer, optionally after activating the surface to provide pendant reactive groups, as described above.

The polymer may further act as a drug delivery vehicle, for instance it may be preloaded with a pharmaceutically active compound and act as a reservoir for release over an extended period. Suitable actives are anti-tumour compounds, such as cytotoxic compounds, or anti-inflammatories such as cortico-steroids. One example of a cortico-steroid which has been incorporated into polymeric drug delivery implants is dexamethasone. It is believed that such drugs may increase the sensitivity of the proliferating cells to apoptotic signalling, and thus act synergistically in the present invention (Evans. Storm et al. J. Steroid Biochem. Mol. Biol. (1995) 53.18).

In another aspect of the invention there is provided a reactive conjugate of an oligomer and a DRligand in which the oligomer is bound to the DRligand by a biostable linkage, and at a site on the DRligand such that the DR binding domain of the ligand remains biologically active, and substantially unimpeded in its DR binding. This may be determined by binding tests. The reactive group is on the oligomer portion of the conjugate, and is suitable for enabling the conjugate to be attached to a substrate, usually the surface of a solid polymer, for instance the surface of a medical device.

The invention is illustrated in the accompanying example:

Materials

Fas ligand used in the liquid state examples is anti-Fas IgM antibody, clone CH II from UpState Biotechnology. The Fas ligand immobilised onto the polymer is a derivative of natural Fas ligand and available from Alexis Biochemicals.

### EXAMPLE 1 (REFERENCE)

Purpose: To investigate death receptor-mediated human lens epithelial cell (HLE) apoptosis. Methods: The method for determining the presence of Fas receptor on the surface of lens epithelial cells is based on immunohistochemistry and western blotting based on Wilson et al., op. cit. Generally the method is carried out as follows. Cell extracts are size separated by gel electrophoresis and transferred electrophoretically to a nitrocellulose membrane. The membrane is than incubated with antibodies against Fas, and bound enzymes are detected by an enzyme linked assay (Gershoni J M, Palade G E. Protein blotting: principles and applications. Analyt Biochem 1983; 131: 1-15). To determine the spacial distribution of DR expression, histological preparations of LECs are bound by fluorescent labled antibodies to DR. Death receptor (Fas (CD95), TNF-R1 and DR-4) expression was evaluated by western blot analysis in an established lens epithelial cell line (SRA 01/04)). LEC monolayers (n=6 for each treatment) were then treated with either anti-Fas IgM (CH11) 50 ng/ml and 500 ng/ml, control IgM 50 ng/ml and 500 ng/ml, or left untreated. Monolayers were also treated with 5-fluorouracil 12.5 mg/ml, which served as a positive control for inducing LEC apoptosis. After 48 hours LEC death was quantified using a lactate dehydrogenase release assay. The mode of cell death was ascertained by evaluating cell morphology cytospin preparations by light microscopy. Results: The results indicate that treatment of LEC monolayers with anti-Fas IgM for 48 hours resulted in significant cell death, compared to control IgM or untreated controls. Light microscopy revealed condensation and fragmentation of nuclear chromatin, characteristic of apoptotic cell death.

TABLE 1

| Treatment | Lactate dehydrogenase release |
|---|---|
| Anti-Fas IgM | 22.4% +/− 5.3 |
| Control IgM | 1.2% +/− 2.9 |

Anti-Fas IgM and 5-fluorouracil consistently led to significantly greater levels of LEC death at 48 hours than untreated controls (22.4%+/−5.3 P<0.01). Control IgM had no effect on cell viability compared to untreated controls (1.2%+/−2.9). Anti Fas-IgM and 5-fluorouracil induced condensation and fragmentation nuclear chromatin which are hallmarks of apoptotic death. Western blot analysis confirmed Fas receptor expression. We were unable to detect TNF-R1 and DR-4 expression. We conclude that active Fas ligand induces apoptosis in this LEC cell line.

### EXAMPLE 2

A poly(2-hydroxy ethyl methacrylate-cross-linker) membrane of the type used in IOL's is derivatised with Fas ligand and tested for the effect on Jurkatt T cells, known Fas-sensitive cells (Ponton, A et al. J. Biol. Chem. (1996) 271, 8991-8995). First the pHEMA membrane was derivatised by reacting with a heterobifunctional PEG linker precursor. The precursor had one of its reactive groups in protected form and the other activated to conjugate to hydroxyl groups at the surface. The intermediate polymer-linker conjugate was then activated and conjugated to Fas ligand (Alexis Biochemicals). Excess reactive groups at the surface were then deactivated

Jurkatt T cells were seeded at 2×10⁴ cells/well in a 96 well plate in MEM without phenol red, containing 0.1% fatty acid free BSA. Cells were either untreated, treated with CH11 (anti-Fas IgM, Upstate Biotechnology) at 100 ng/ml, or incubated with a square of immobilised Fas ligand placed in the bottom of the well. To assay LDH release, medium samples were centrifuged to pellet cells and the supernatant assayed for LDH release using the Cytotoxicity Detection Kit (Roche), according to the manufacturers instructions. Total LDH release was determined by solubilising cells with 1% TX100 prior to centrifugation and LDH assay.

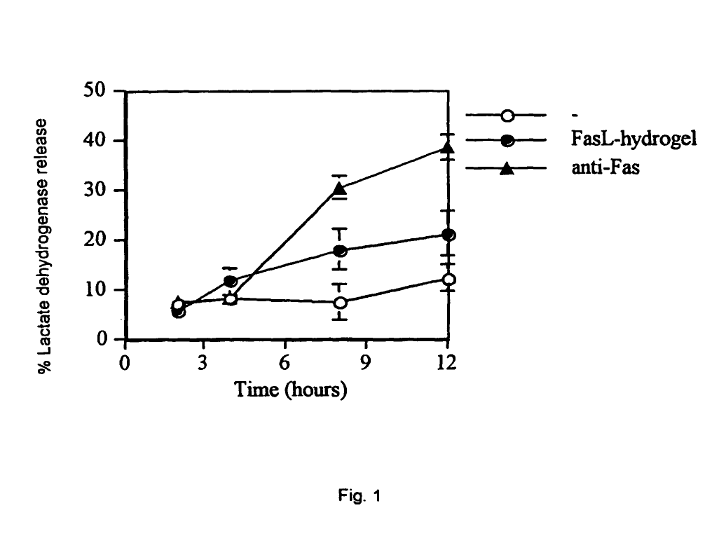

The death surface (Hydrogel-PEG-FAS ligand) produced increased cell death in Jurkatt T cells (a well recognised Fas sensitive control), compared to untreated controls (see FIG. 1) which shows % lactate dehydrogenase release against time. Unbound Fas stimulating antibody produced a greater effect.

1. Use of a product comprising death receptor ligand covalently bound to a polymer in the manufacture of a composition for use in the treatment of a human or animal.

2. Use according to claim 1 in which the polymer is at the surface of a solid substrate.

3. Use according to claim 1 or claim 2 in which the ligand is bound to the polymer through a spacer.

4. Use according to any preceding claim in which the ligand recognises Fas.

5. Product according to claim 4 in which the ligand is bi- or higher valent Fas antibody, or is Fas ligand.

6. Use according to any preceding claim in which the polymer is a hydrogel or is elastomeric.

7. Use according to claim 6 in which the polymer is silicone.

8. Use according to any preceding claim in which the product which is an intraocular lens.

9. Use according to any of claims 1 to 7 in which the product is a wound dressing.

10. Use according to any preceding claim in which, in the method of treatment, the polymer is contacted with cells expressing the corresponding death receptor at the cell surface.

11. Use according to claim 10 in which the cells are epithelial cells.

12. Method in which a product comprising death receptor ligand covalently bound to a polymer is contacted in vitro with cells expressing the corresponding death receptor at the cell surface.
